(19) United States
(12) Patent Application Publication
(10) Pub. No.: US 2023/0000425 A1
(43) Pub. Date: Jan. 5, 2023

(54) SKIN DIAGNOSING DEVICE

(71) Applicant: LG ELECTRONICS INC., Seoul (KR)

(72) Inventors: Changkyu HWANG, Seoul (KR); Sungkwon JANG, Seoul (KR); Juyeon SHIN, Seoul (KR); Seong JO, Seoul (KR); Kwangjun AN, Seoul (KR)

(73) Assignee: LG ELECTRONICS INC., Seoul (KR)

(21) Appl. No.: 17/781,994

(22) PCT Filed: Jan. 16, 2020
(86) PCT No.: PCT/KR2020/000774

§ 371 Date: Jun. 2, 2022

(30) Foreign Application Priority Data
Dec. 6, 2019 (KR) 10-2019-0161610

(51) Int. Cl. A61B 5/00

(52) U.S. Cl. CPC A61B 5/442; A61B 5/0077; A61B 5/443

(57) ABSTRACT

A skin diagnosing device includes a main body provided with an input interface for operation; and a head connected to one end of the main body, the head includes: a front cover forming a contact surface with of a user and including an opening; a cover window covering the opening; a plurality of ultraviolet light sources and a plurality of visible light sources, which are disposed behind the front cover and face the opening; at least one camera which is disposed behind the plurality of ultraviolet light sources and the plurality of visible light sources, and faces the opening; and a laser light source which is disposed behind the at least one camera and faces the opening.

### TECHNICAL FIELD

The present invention relates to a skin diagnosing device.

### BACKGROUND ART

The skin care is aimed at maintenance of blemish-free, clean, and smooth skin, and in particular, the greatest interest has been in caring the facial skin among the body parts. Therefore, people try to keep their clean skin by receiving massage, applying a functional cosmetic product, or using various cleaning products to care for the facial skin.

The people's skin is largely classified into dry, neutral, and oily, and specifically, since the people's skin is variously classified according to conditions such as oil and moisture, color tone, elasticity, etc., the use of appropriate products or skin care methods according to respective types may be required.

In the case of various skin diagnosing devices or systems according to the related art, most of the skin diagnosing devices or systems are large and expensive products, and as the skin diagnosing devices or systems require use by experts, there is a limitation in providing the skin diagnosing devices or systems at limited places such as hospitals.

Thus, in general, since people select a product or skin care method by determining a skin type based on their subjective judgment, a problem of worsening skin conditions may occur due to misuse of the product or incorrect skin care.

### DISCLOSURE OF THE INVENTION

### Technical Problem

An object of the present invention is to provide a skin diagnosing device having a compact size while having various components for diagnosing a skin condition.

Another object of the present invention is to provide a skin diagnosing device capable of uniformly measuring and diagnosing a skin diagnosis area while having a compact size.

### Technical Solution

A skin diagnosing device according to an embodiment of the present invention includes a main body provided with an input interface for an operation and a head connected to one end of the main body, wherein the head includes a front cover configured to form a contact surface and including an opening, a cover window configured to cover the opening, a plurality of ultraviolet light sources and a plurality of visible light sources, which are disposed behind the front cover and face the opening, at least one camera which is disposed behind the plurality of ultraviolet light sources and the plurality of visible light sources and faces the opening, and a laser light source which is disposed behind the at least one camera and faces the opening.

The skin diagnosing device may further include a first inner body in which the plurality of ultraviolet light sources are provided and which is coupled to a rear side of the front cover and a second inner body in which the plurality of visible light sources are provided and which is coupled to a rear side of the first inner body, wherein the first inner body may include an opening formed in an area including a center the first inner body, and each of the plurality of ultraviolet light sources may be spaced a first distance from the center of the first inner body.

Each of the plurality of visible light sources may be spaced a second distance greater than the first distance from a center of the second inner body, the opening of the first inner body may include a main opening formed in an area including the center and a plurality of sub openings formed outside the main opening, and a radius of the main opening may be less than the second distance.

In an embodiment, each of the plurality of sub openings may be formed as an area between the two ultraviolet light sources adjacent to each other.

In an embodiment, the second distance is a distance for avoiding provision of light reflected from the cover window in light emitted from the plurality of visible light sources to a diagnosis area that is preset within an image acquired through the at least one camera.

The second inner body may include a camera opening formed in the area including the center and a laser light source opening formed in an area between the laser light source and the cover window on an area between the camera opening and an edge of the second inner body.

The skin diagnosing device may further include a polarization assembly including a first polarizing film formed in front of some of the plurality of visible light sources and a second polarizing film formed in front of the rest of the plurality of visible light sources.

Any one of the first polarizing film and the second polarizing film may be formed in front of any one camera of the at least one camera.

In an embodiment, a distance between a central axis of the head in a front and rear direction and the at least one camera may be less than that between the central axis and the laser light source. In this case, the laser light source may be disposed to be inclined at a predetermined angle with respect to the front and rear direction of the head.

A radius of an inner circumferential surface of the front cover corresponding to the opening may be less than each of the first distance and the second distance.

The radius of the inner circumferential surface of the front cover may gradually decrease toward the contact surface of the front cover.

In an embodiment, the head may be formed to extend from one end of the main body at an obtuse angle.

### Advantageous Effects

According to an embodiment of the present invention, the various components such as the ultraviolet light source, the visible light source, the camera, and the laser light source may be provided in the head of the skin diagnosing device to provide the various diagnosis information related to the user's skin condition.

In addition, the skin diagnosing device may implement the head having the compact size by disposing the laser light source at the position that is furthest from the front cover and disposing the remaining components between the laser light source and the front cover based on the optical characteristics.

In addition, the visible light source provided in the skin diagnosing device may be disposed to avoid the reflected light from being provided to the diagnosis area in the image acquired by the camera to acquire more accurate diagnosis information about the skin.

In addition, the chamfer structure may be formed on the inner circumferential surface of the front cover to minimize the shaded area generated inward from the edge of the opening. Therefore, since the shaded area is provided on the diagnosis area in the image acquired by the camera, the possibility of generating the inaccurate diagnosis information may be prevented in advance.

### BRIEF DESCRIPTION OF THE DRAWINGS

FIG. 1 is a perspective view of a skin diagnosing device when viewed from one side according to an embodiment of the present invention.

FIG. 2 is a perspective view of the skin diagnosing device when viewed from the other side according to an embodiment of the present invention.

FIG. 3 is an exploded perspective view illustrating a head of the skin diagnosing device.

FIG. 4 is a cross-sectional view illustrating the head of the skin diagnosing device.

FIG. 5 is a view illustrating a light irradiation area of a laser light source and a skin diagnosis area in an image acquired by a camera, which are disposed according to the embodiment of FIG. 4.

FIGS. 6a to 7b are exemplary views for explaining an arrangement of a second light source for preventing reflected light from being provided to the skin diagnosis area of the image acquired by the camera.

FIG. 8 is a front view of a second light source assembly including a second light source disposed according to FIGS. 7a to 7b.

FIG. 9 is a front view of a first light source assembly disposed in front of the second light source assembly.

FIG. 10 is a front view illustrating a state in which a front cover and a side cover of the skin diagnosis device are removed according to an embodiment of the present invention.

FIG. 11a to 12b are views for explaining a shape of the front cover for preventing a shaded area is provided on the skin diagnosis area of the image acquired by the camera.

### MODE FOR CARRYING OUT THE INVENTION

Hereinafter, embodiments disclosed in this specification is described with reference to the accompanying drawings, and the same or corresponding components are given with the same drawing number regardless of reference number, and their duplicated description will be omitted. Furthermore, terms, such as a “module” ad a “unit”, are used for convenience of description, and they do not have different meanings or functions in themselves. Moreover, detailed descriptions related to well-known functions or configurations will be ruled out in order not to unnecessarily obscure subject matters of this specification. However, this does not limit this specification within specific embodiments and it should be understood that the present disclosure covers all the modifications, equivalents, and replacements within the idea and technical scope of this specification.

It will be understood that although the ordinal numbers such as first and second are used herein to describe various elements, these elements should not be limited by these numbers. The terms are only used to distinguish one component from other components.

It will also be understood that when an element is referred to as being “'connected to” or “engaged with” another element, it can be directly connected to the other element, or intervening elements may also be present. It will also be understood that when an element is referred to as being ‘directly connected to’ another element, there is no intervening elements.

The terms of a singular form may include plural forms unless referred to the contrary.

In this application, the terms “comprises” or “having” are intended to indicate that there is a feature, number, step, operation, component, part, or combination thereof described in the specification, and one or more other features. It is to be understood that the present invention does not exclude the possibility of the presence or the addition of numbers, steps, operations, components, components, or a combination thereof.

Hereinafter, exemplary embodiments of the present disclosure will be described in detail with reference to the accompanying drawings.

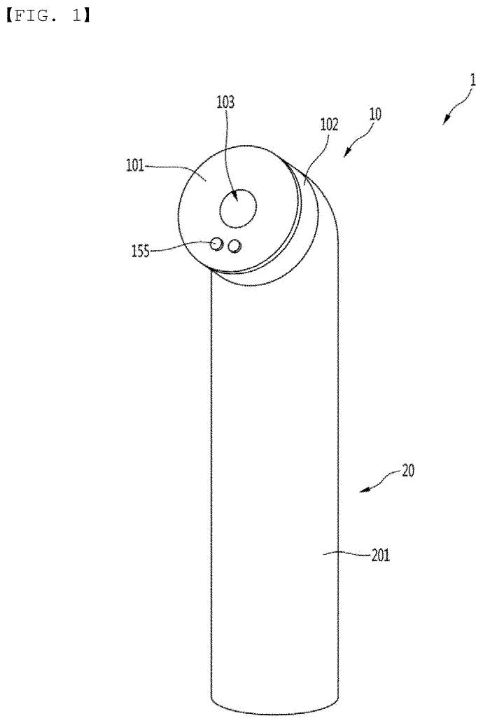

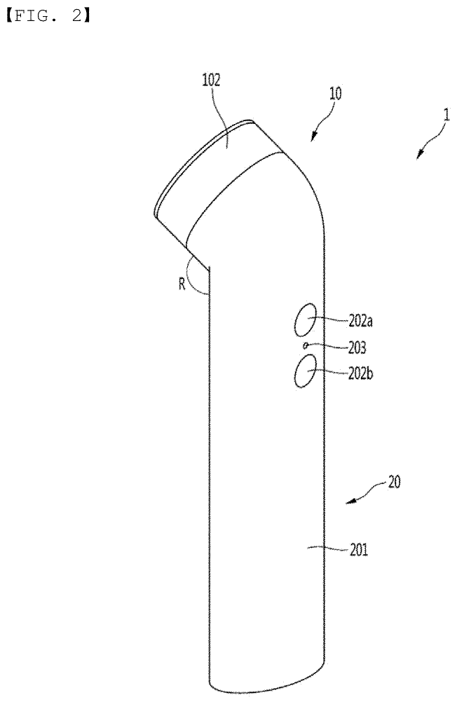

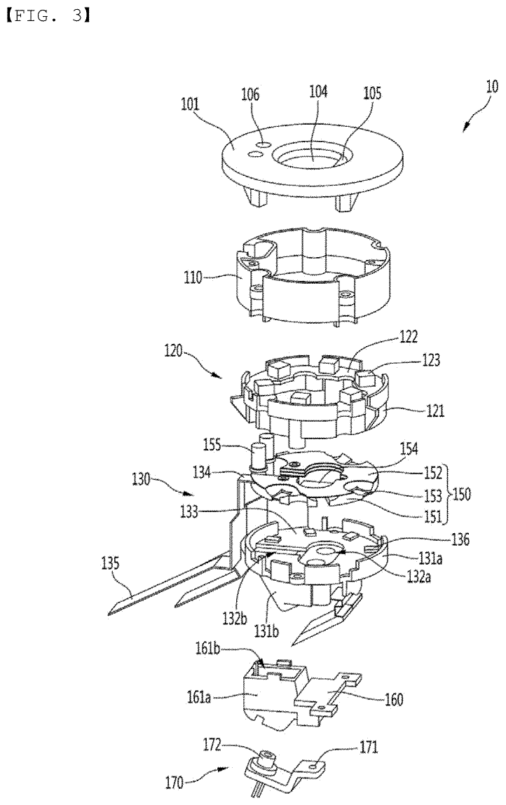

FIG. 1 is a perspective view of a skin diagnosing device when viewed from one side according to an embodiment of the present invention, and FIG. 2 is a perspective view of the skin diagnosing device when viewed from the other side according to an embodiment of the present invention.

Referring to FIGS. 1 and 2, a skin diagnosing device 1 according to an embodiment of the present invention may be a device for acquiring various data related to user's skin conditions. The skin diagnosing device 1 may include a head 10 connected to a main body 20 and the main body 20 for user's gripping.

The user allows the head 10 to be in contact with the user's skin while holding the main body 20 so that the skin diagnosing device 1 acquires various data related to the skin conditions. In order to allow the head 10 to be in easily contact with the sink by the user while holding the main body 20, the head 10 may be formed to extend from one end of the main body 20 at a predetermined angle R (e.g., an obtuse angle). For example, the angle R may be 120°, but is not limited thereto.

The head 10 may include a front cover 101 that forms a contact surface with the skin. In addition, the head 10 may include a side cover 102 that covers a side surface.

Components for acquiring the data related to the skin conditions may be provided in an inner space of the head 10, which is formed by the front cover 101 and the side cover 102.

An opening 103 through which light emitted from a light source disposed in the front cover 101 is irradiated to the skin to acquire an image of the skin by a camera disposed in the front cover 101 may be formed in the front cover 101. That is, the skin diagnosing device 1 may acquire image data for skin diagnosis through the light source and the camera.

The front cover 101 and the opening 103 may generally have a circular shape, but is not limited thereto.

The opening 103 may be formed in an area including a center of the front cover 101, and the front cover 101 may be in close contact with the skin. Thus, it is possible to prevent the light emitted from the light source from leaking to the outside or prevent external light from being introduced to prevent an inaccurate image of the skin from being acquired.

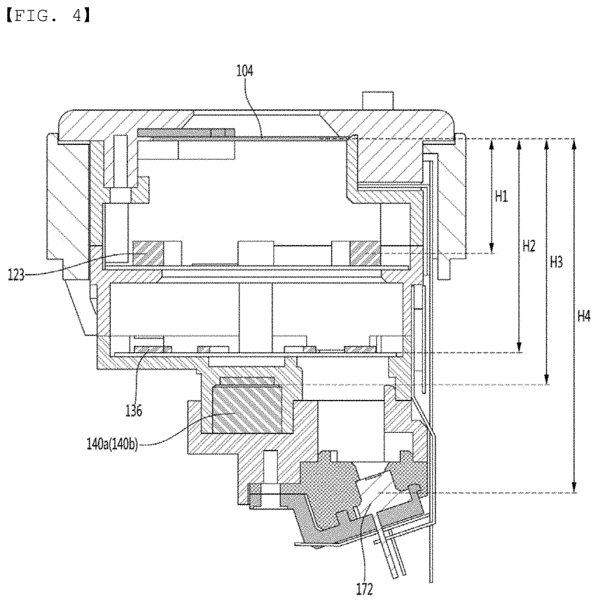

A sensor hole 106 (see FIG. 3) through which one end of a moisture measuring sensor 155 disposed inside the head 10 is exposed may be formed in the front cover 101. The skin diagnosing device 1 may acquire sensing data related to a moisture level of the skin as the moisture measuring sensor 155 is in contact with the skin.

According to an embodiment, when the contact of the front cover 101 with the skin is detected based on the sensing data of the moisture measuring sensor 155, the skin diagnosing device 1 may drives the light source and the camera, which are disposed therein, acquire the data (image data and/or sensing data of the moisture measuring sensor) related to the skin conditions. Thus, it is possible to prevent side effects due to the irradiation of internal ultraviolet light or laser light to the user's eyes or the like from occurring.

The main body 20 may include a case 201 that forms an outer appearance. The case 201 is formed to have a substantially cylindrical shape so that the user easily grasps the case 201 by his/her hands.

Although not shown, a main board on which components (processor, memory, etc.) for controlling driving of the components provided in the head 10 or controlling power supply are mounted may be provided inside the main body 20.

At least one input interface (e.g., buttons 202a and 202b) for turning on/off the skin diagnosing device 1 or setting an operation mode may be provided on one surface of the case 201. Also, an indicator 203 (e.g., the light source) for notifying a power state or an operation state of the skin diagnosing device 1 may be provided on one surface of the case 201.

A connection terminal connected to other electronic devices (tablet PC, smart phone, PC, etc.) may be formed in a lower portion of the main body 20. In this case, the skin diagnosing device 1 may be driven by receiving power from an electronic device connected through the connection terminal and may transmit data (image data and/or sensing data of the moisture measuring sensor) acquired in relation to the skin conditions to the electronic device. That is, a separate battery may not be provided inside the main body 20, and thus a weight of the skin diagnosing device 1 may be minimized.

In this case, the data acquired by the skin diagnosing device 1 may be analyzed by the electronic device connected to the skin diagnosing device 1. That is, the electronic device may acquire and provide user's skin diagnosis information based on the data (such as image data and/or sensing data of the moisture measuring sensor) acquired from the skin diagnosing device 1.

However, a battery may be provided inside the main body 20 according to an embodiment.

Hereinafter, various embodiments related to the head 10 of the skin diagnosing device 1 will be described with reference to FIGS. 3 to 12b.

FIG. 3 is an exploded perspective view illustrating a head of the skin diagnosing device.

In the following drawings, a direction in which the front cover 101 is disposed with respect to the second light source assembly 130 is defined as a front side of the head 10, and a direction in which the laser light source assembly 170 is disposed is defined as a rear side of the head 10.

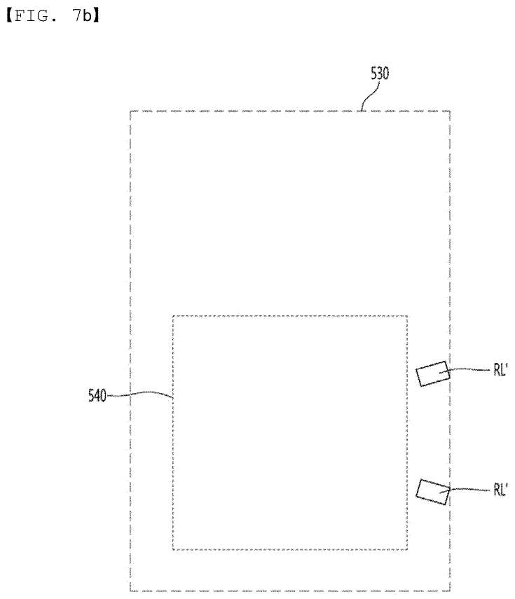

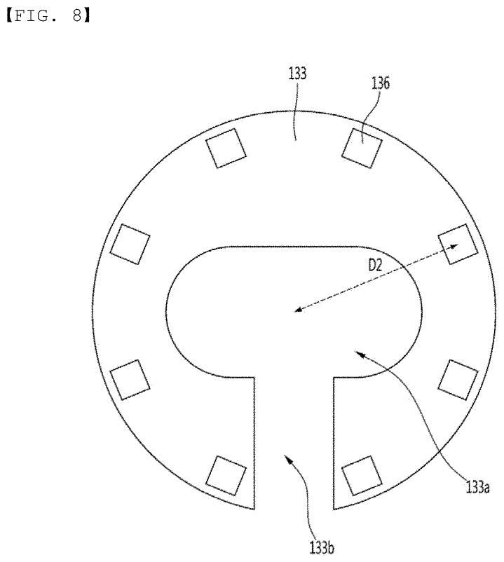

Referring to FIG. 3, the head 10 may include the front cover 101, a first light source assembly 120 disposed behind the front cover 101, a second light source assembly 130 disposed behind the first light source assembly 120, a plurality of cameras 140a and 140b (see FIG. 7a) disposed behind the second light source 136, and a laser light source assembly 170 disposed behind the plurality of cameras 140a and 140b.

The front cover 101 may cover a front surface of the head 10 and form a contact surface with the skin. As described with reference to FIGS. 1 to 2, the opening 103 may be formed in the front cover 101.

As the opening 103 is formed, there is a risk that external dust, foreign matters, moisture, etc. may be penetrated into the inside of the head 10 through the opening 103. To prevent this, the head 10 may include a cover window 104 that covers the opening 103 to block the inside and the outside of the head 10.

The cover window 104 may be coupled to a rear side of the front cover 101 or fixed between the front cover 101 and a front inner body 110.

The cover window 104 may be made of a material such as transparent glass or plastic. Thus, the light emitted from the light source provided inside the head 10 may pass through the cover window 104 and then be irradiated to the skin, and light reflected from the skin may pass through the cover window 104 and then be incident into the cameras 140a and 140b.

The head 10 may further include a gray card 105 formed to correspond to a partial area of the opening 103.

The gray card 105 may be provided to acquire more accurate image data by correcting camera deviations of the skin diagnosing devices. The skin diagnosing device 1 or a processor of the electronic device connected to the skin diagnosing device 1 may correct a color deviation with respect to the image on other areas (skin diagnosis area), based on the image on an area corresponding to the gray card 105 among the images acquired through the cameras 140a and 140b.

The gray card 105 may be disposed in front of the cover window 104 to minimize a distance from the skin, thereby enabling more accurate correction of the image. However, the gray card 105 may be disposed behind the cover window 104 according to an embodiment.

The first light source assembly 120 may be disposed behind the front cover 101. A front inner body 110 through the light is uniformly irradiated to the areas of the skin by securing a sufficient separation distance between the cover window 104 and the first light source 123 may be provided the front cover 101 and the first light source assembly 120.

The front inner body 110 may be formed in a ring shape having a predetermined length in a front and rear direction. Thus, the sufficient separation distance may be formed between the front cover 101 and the first light source assembly 120. In addition, light emitted from the internal light sources may pass through the opening of the front inner body 110, and light reflected from the skin may be incident into the camera. The front inner body 110 may be coupled to the front cover 101 through a coupling member such as a screw.

The first light source assembly 120 may be coupled to a rear side of the front inner body 110.

The first light source assembly 120 may include a middle inner body 121, a first light source mounting plate 122 coupled to a front side of the middle inner body 121, and a plurality of first light sources 123 mounted on a front side of the first light source mounting plate 122.

The middle inner body 121 may be coupled to the front inner body 110 to fix the first light source mounting plate 122 and the plurality of first light sources 123.

Each of the middle inner body 121 and the first light source mounting plate 122 may have a ring shape. Thus, an opening 124a may be formed in an area including a center of each of the middle inner body 121 and the first light source mounting plate 122.

Light emitted from the second light source 136 and the laser light source 172 disposed behind the first light source assembly 120 may pass through the cover window 104 through the opening 124a and then be irradiated to the skin. In addition, light reflected from the skin may be incident into the cameras 140a and 140b through the opening 124a.

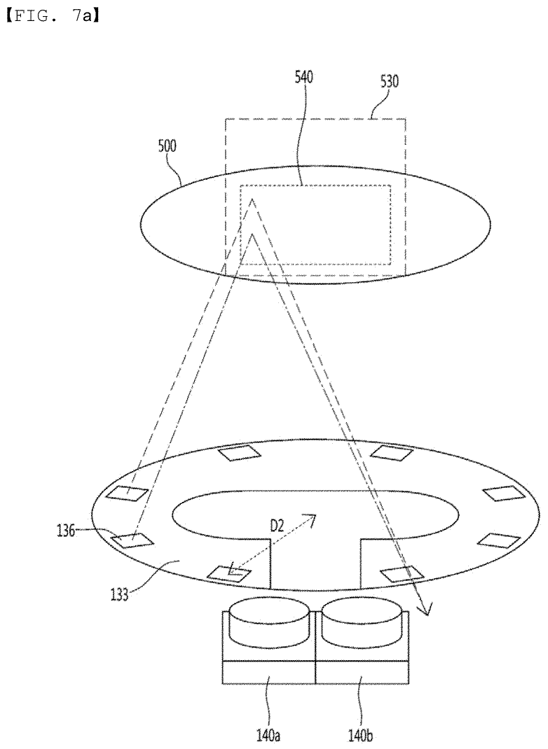

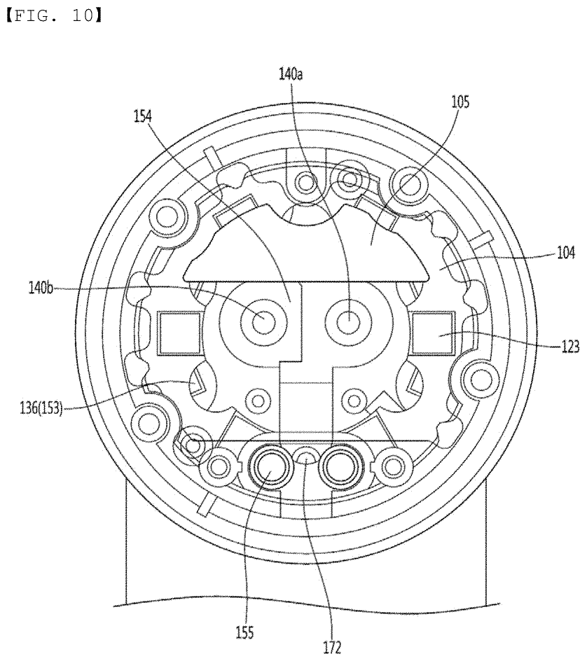

According to an embodiment, a plurality of sub openings 124b (see FIG. 9) may be further formed outside the opening 124a. In this case, the opening 124a may be defined as a main opening 124a. Contents related to the main opening 124a and the sub opening 124b will be described later with reference to FIGS. 6a to 10.

The plurality of first light sources 123 may be disposed to face the front cover 101. For example, the plurality of first light sources 123 may be implemented as ultraviolet light sources emitting ultraviolet light. The ultraviolet light source may include a UV-LED that emits light having a UV-A wavelength.

According to an embodiment, since the plurality of first light sources 123 are spaced regular intervals from each other to minimize a deviation for each area of the light irradiated to the skin.

The second light source assembly 130 may be coupled to a rear side of the first light source assembly 120.

The second light source assembly 130 may include rear inner bodies 131a and 131b, a second light source mounting plate 133 coupled to a front side of each of the rear inner bodies 131a and 131b, a plurality of second light sources 136 mounted on the second light source mounting plate 133, and a polarization assembly 150 coupled to a front side of the second light source mounting plate 133.

The rear inner bodies 131a and 131b may include a plate mounting body 131a forming a mounting surface of the second light source mounting plate 133 and a camera accommodating body 131b extending to a rear side of the plate mounting body 131a to form an accommodating space that accommodates the cameras 140a and 140b.

The plate mounting body 131a may have a substantially disk shape. A camera opening 132a formed to correspond to the cameras 140a and 140b and a laser light source opening 132b formed to correspond to the laser light source 172 may be formed in the plate mounting body 131a.

As illustrated in the drawings, at least one camera opening 132a may be formed to correspond to each of at least one camera 140a and 140b, but this is not always limited thereto, and one opening may be formed regardless of the number of cameras.

The laser light source opening 132b may be formed to correspond to the opening 161b formed in a laser light source fixing body 160 to be described later.

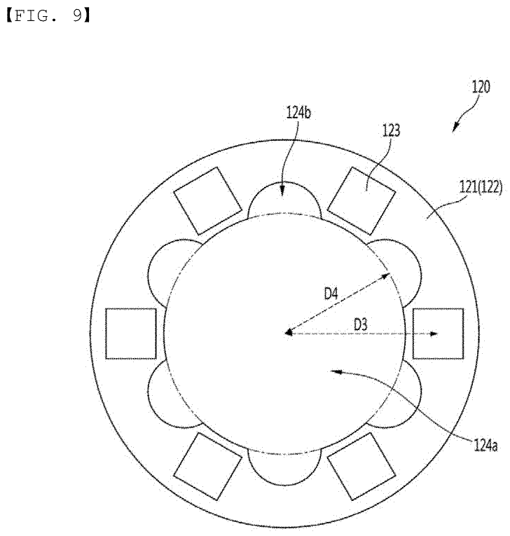

The second light source mounting plate 133 may be mounted in front of the plate mounting body 131a. A first opening 133a (see FIG. 8) corresponding to the camera opening 132a and a second opening 133b (see FIG. 8) corresponding to the laser light source opening 132b may be formed in the second light source mounting plate 133. For example, a surface area of the first opening 133a may be equal to or greater than that of the camera opening 132a, and a surface area of the second opening 133b may be equal to or greater than that of the laser light source opening 132b, but are limited thereto.

The second light source mounting plate 133 may be implemented as a circuit board, but this is not necessarily limited thereto. When the second light source mounting plate 133 is the board, the second light source mounting plate 133 may be integrally formed with connection boards 134 and 135 or may be connected to the connection boards 134 and 135. The connection boards 134 and 135 may be connected to the main body 20 to transmit signals or power to the first light source 123, the second light source 136, the cameras 140a and 140b, the moisture measuring sensor 155, and the laser light source.

A plurality of second light sources 136 disposed to face the front cover 101 may be mounted on a front surface of the second light source mounting plate 133. For example, the plurality of second light sources 136 may be implemented as a visible light source (e.g., white light source) emitting visible light. The white light source may be implemented as an LED emitting white light.

According to an embodiment, the above-described first light source 123 may be implemented as the visible light source, and the second light source 136 may be implemented as the ultraviolet light source.

Since the plurality of second light sources 136 are spaced regular intervals from each other to minimize a deviation for each area of the light irradiated to the skin.

The cameras 140a and 140b may be accommodated in the camera accommodating body 131b and disposed to face the front cover 101.

For example, the skin diagnosing device 1 may include two cameras 140a and 140b arranged side by side. In this case, a camera polarizing film 154 to be described later may be disposed in front of any one camera.

The polarization assembly 150 may be provided in front of the rear inner bodies 131a and 131b. The polarization assembly 150 may include a polarizing body 151 and an upper polarizing film 152 and a lower polarizing film 153, which are fixed to both surfaces of the polarizing body 151, respectively.

The polarizing body 151 may have a shape generally corresponding to the second light source mounting plate 133. That is, openings corresponding to the first openings 133a and the second openings 133b may be formed in a center of the polarizing body 151.

In addition, a plurality of light source grooves formed at positions corresponding to the plurality of second light sources 136 may be formed in an edge of the polarizing body 151. The upper polarizing film 152 or the lower polarizing film 153 may be disposed in each of the plurality of light source grooves.

The upper polarizing film 152 and the lower polarizing film 153 may transmit polarized light obtained by filtering any one of a vertical component and a horizontal component of the light emitted from the second light source 136.

The upper polarizing film 152 and the lower polarizing film 153 may be implemented as different types of polarizing films. For example, the upper polarizing film 152 may be implemented as a horizontal polarizing film, and the lower polarizing film 153 may be implemented as a vertical polarizing film. That is, some of the plurality of second light sources 136 may be implemented to radiate horizontally polarized light to the skin, and others may be implemented to radiate vertically polarized light to the skin.

The lower polarizing film 153 (or the upper polarizing film 152) may extend to an area corresponding to the front of any one of the at least one camera 140a or 140b to form the camera polarizing film 154. In this case, any one of the cameras (e.g., the second camera 140b) may function as a polarizing camera.

The skin diagnosing device 1 may acquire various images of the skin through the plurality of second light sources 136 and the cameras 140a and 140b. That is, the skin diagnosing device 1 or the processor (not shown) of the electronic device connected to the skin diagnosing device 1 may drive some of the plurality of second light sources 136 based on types of skin diagnosis information to be acquired and may drive any one of the first camera 140a and the second camera 140b (assuming a polarizing camera).

According to an embodiment, the moisture measuring sensor 155 may be provided in front of the plate mounting body 131a. The moisture measuring sensor 155 may be partially exposed to the front side of the front cover 101 through the sensor hole 106. For example, the moisture measuring sensor 155 may be implemented as a current sensor that measures current when being in contact with the skin, and in this case, the sensing data may correspond to a current measurement value. As an amount of moisture in the skin increases, the current measurement value may increase.

The laser light source assembly 170 may be disposed behind the second light source assembly 130. For example, the laser light source assembly 170 may be fixed to a rear side of the second light source assembly 130 through a laser light source fixing body 160.

The laser light source fixing body 160 may include a laser light source accommodating part 161a that forms an accommodating space of the laser light source 172 and an opening 161b formed in front of the laser light source accommodating part 161a.

The laser light source assembly 170 may include a coupling body 171 for coupling the laser light source 172 to the laser light source fixing body 160. The coupling body 171 may be coupled to a rear side of the laser light source fixing body 160.

The cameras 140a and 140b may be disposed in front of the laser light source 172, and the cameras 140a and 140b may be disposed adjacent to a central axis of the head 10 in the front and rear direction. Thus, the laser light source 172 may be disposed on an edge area spaced apart from the central axis and may be disposed to be inclined at a predetermined angle with respect to the front and rear direction of the head 10 in order to irradiate the laser light to the cover window 104.

The laser light source 172 may include a laser diode that emits laser light.

According to an embodiment of the present invention, the skin diagnosing device 1 may emit any one of the ultraviolet light, the visible light (white light), and the laser light, which corresponds to the skin diagnosis information to be acquired and may acquire the skin diagnosis information based on the image data acquired through any one camera. That is, the skin diagnosing device 1 is implemented to acquire diagnosis information on various skin indicators (oil, skin tone, pores, wrinkles, pigmentation, elasticity, sebum, etc.).

Specifically, when it is desired to acquire the diagnosis information on the oil, the skin tone, the pores, and the wrinkles of the skin, the skin diagnosing device 1 may turns on the plurality of second light sources 136 and acquire image data through a general camera (e.g., the first camera 140a).

In addition, when it is desired to acquire the diagnosis information on the skin pigmentation, etc., the skin diagnosing device 1 may turn on the second light sources disposed behind the vertical polarizing film among the plurality of second light sources 136 to acquire vertically polarized image data.

In addition, the skin diagnosing device 1 may turn on the laser light source 172 to acquire diagnosis information on the elasticity of the skin and also turn on the ultraviolet light source 123 to acquire the diagnosis information on the sebum of the skin.

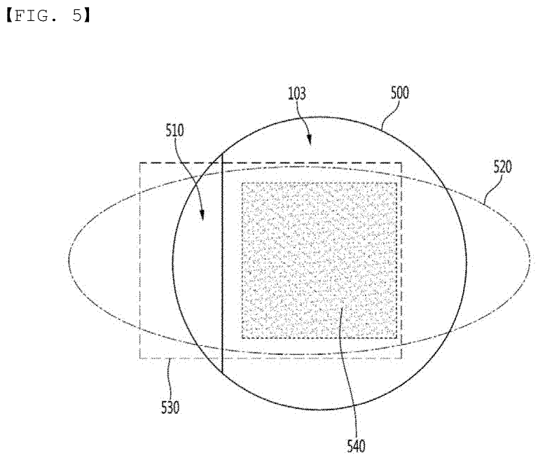

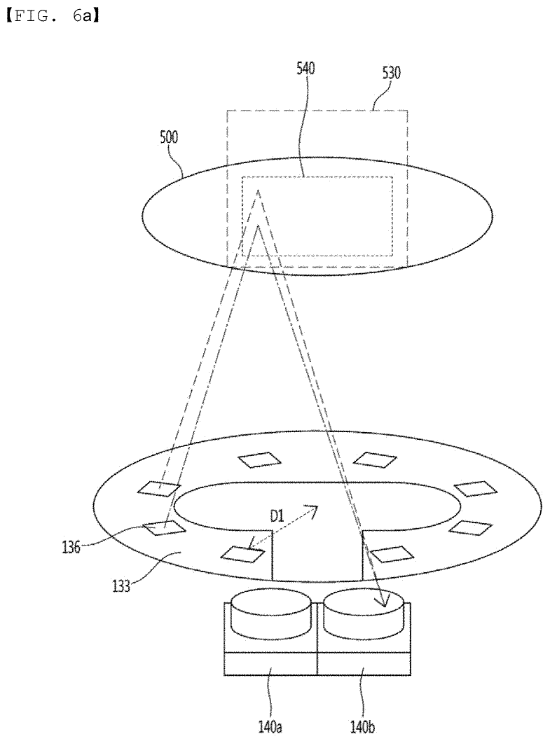

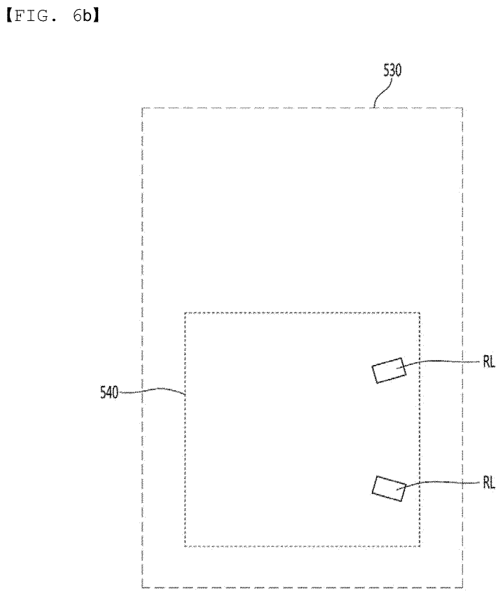

FIG. 4 is a cross-sectional view illustrating the head of the skin diagnosing device, and FIG. 5 is a view illustrating a light irradiation area of a laser light source and a skin diagnosis area in an image acquired by a camera, which are disposed according to the embodiment of FIG. 4.

Referring to FIGS. 4 and 5, when the front cover 101 is in contact with the skin, skin diagnosis information on an area 500 corresponding to the opening 103 may be acquired.

In order to acquire accurate diagnosis information on the skin, data (image data) on an area having a predetermined size or more (hereinafter, referred to as a ‘diagnosis area’) of the area 500 may be required. In addition, image data for a correction area 510 corresponding to the gray card 105 may be required to correct a deviation of the light sources or a deviation of the cameras between products.

In the case of the ultraviolet light source (e.g., the first light source 123) and the visible light source (e.g., the second light source 136), a plurality of light sources may be provided, and since straightness of light is relatively low, the light may be evenly irradiated to the area 500.

On the other hand, in the case of the laser light source 172, since straightness of the laser light is relatively higher than that of the ultraviolet light source and the visible light source, a distance from the skin (or the cover window 104) has to be long to evenly irradiate the light to the diagnosis area and the correction area 510.

However, in order to implement the skin diagnosing device 1 having a compact size, a length of the head 10 may be limited within a predetermined length.

Based on this, the laser light source 172 may be disposed to be maximally spaced apart from the cover window 104 based on the structure, length, or size of the head 10. For example, when the laser light source 172 is disposed to have a predetermined distance H4 from the cover window 104, a laser light irradiation area 520 to which the laser light is uniformly irradiated may be formed. Thus, a diagnosis area 540 for acquiring the diagnosis information of the skin may be secured.

The cameras 140a and 140b may be spaced a predetermined distance H3 from the cover window 104 to form a photographing area 530 including the correction area 510 and the diagnosis area 540.

Based on the positions of the cameras 140a and 140b, the plurality of first light sources 123 and the plurality of second light sources 136 may be disposed between the cameras 140a and 140b and the cover window 104 to minimize an increase in size of the skin diagnosing device and minimize a decrease in amount of light due to the increase in separation distance.

That is, the distance H4 between the laser light source 172 and the cover window 104 may be greater than a distance H1 between the first light source 123 and the cover window 104, a distance H2 between the second light source 136 and the cover window 104, and a distance H3 between each of the cameras 140a and 140b and the cover window 104.

According to the arrangement illustrated in FIG. 4, the skin diagnosing device 1 may acquire and provide the image data of the diagnosis area 540 to which the uniform amount of light is irradiated to provide accurate skin diagnosis information while having the compact size.

FIGS. 6a to 7b are exemplary views for explaining an arrangement of the second light source for preventing reflected light from being provided to the skin diagnosis area of the image acquired by the camera.

Referring to FIG. 6a, a plurality of second light sources 136 (e.g., a white light source) may be disposed on a front surface of the second light source mounting plate 133. When the second light source 136 is turned on to emit light, a portion of the emitted light may pass through the cover window 104 and be irradiated to the skin of the area 500 corresponding to the opening 103. On the other hand, the rest of the emitted light may be reflected by the cover window 104 and be irradiated into the head 10.

Based on this, when the plurality of second light sources 136 are disposed to be spaced a predetermined distance (e.g., a first distance D1) from the center of the second light source mounting plate 133, light reflected from an area corresponding to the diagnosis area 540 of the cover window 104 may be incident into the camera (e.g., the second camera 140b).

In this case, as illustrated in FIG. 6b, at least one reflected light RL may be provided in the diagnosis area 540 on images acquired by the second camera 140b having a predetermined photographing area 530.

As the at least one reflected light RL is provided in the diagnosis area 540, there is a possibility that erroneous diagnosis information on the user's skin may be provided.

Referring to FIG. 7a, the plurality of second light sources 136 according to an embodiment of the present invention may be spaced a second distance D2 from the center of the second light source mounting plate 133 to avoid the generation of the reflected light within the diagnosis area 540.

When the plurality of second light sources 136 are spaced a second distance D2 from the center, the plurality of second light sources 136 may be disposed closer to an edge than a center of the second light source mounting plate 133 (or the second light source assembly 130).

Also, although not shown, the second distance D2 may be greater than a radius of the area 500 formed by the opening 103.

In this case, as illustrated in FIG. 7a, since the light reflected from the area corresponding to the diagnosis area 540 is irradiated to the outside of the second camera 140b, the reflected light. may not be provided on the diagnosis area 540 of the image acquired by the second camera 140b.

Although not shown, when the plurality of second light sources 136 are spaced the second distance D2 from the center, the light reflected from the outer area of the diagnosis area 540 may be irradiated to the second camera 140b. In this case, at least one reflected light RL′ may be provided in the image acquired by the second camera 140b. However, since the at least one reflected light RL′ is provided outside the diagnosis area 540, it may not have any effect in a process of acquiring the diagnosis information.

That is, according to an embodiment illustrated in FIGS. 6a to 7b, the plurality of second light sources 136 may be disposed to be spaced a predetermined distance from the center of the second light source assembly 130 to avoid the provision of the reflected light on the diagnosis area 540, which is previously defined within the image acquired by the cameras 140a and 140b. Thus, an erroneous diagnosis of the skin conditions due to the reflected light being provided in the diagnosis area 540 may be prevented from occurring.

FIG. 8 is a front view of the second light source assembly including the second light source disposed according to FIGS. 7a to 7b, and FIG. 9 is a front view of the first light source assembly disposed in front of the second light source assembly.

Referring to FIGS. 8 to 9, as described above in FIGS. 7a to 7b, the plurality of second light sources 136 may be spaced the second distance D2 from the center of the second light source mounting plate 133 (or the second light source assembly 130) and be disposed adjacent to an edge of the second light source assembly 130 to prevent the reflected light from being provided to the diagnosis area 540.

The first light source assembly 120 is disposed in front of the second light source assembly 130. In addition, the plurality of first light sources 123 may be disposed to be spaced a third distance D3 from the center of the first light source assembly 120.

The third distance D3 may be less than or equal to the second distance D2. When the third distance D3 is greater than the second distance D2, the first light sources 123 are excessively disposed at the edge. Thus, a shaded area may occur within the area 500 of the FIG. 5 due to the thickness of the front cover 101.

When the first light sources 123 are spaced the third distance D3 from each other, since a radius D4 of the opening 124a is formed to be less than the third distance D3, the radius D4 may be less than the second distance D2. Thus, when the plurality of second light sources 136 disposed behind the first light source assembly 120 emit light, a portion of the emitted light may not pass through the opening 124a and be blocked by the rear surface of the first light source assembly 120.

In other words, as the second light sources 136 are disposed to avoid the provision of the reflected light to the diagnosis area 540, a portion of the light emitted from the second light sources 136 may be blocked by the first light source assembly 120 to cause a problem in which an amount of light irradiated onto the skin is reduced.

To prevent this, a plurality of sub openings 124b formed outside the opening 124a may be further formed in the middle inner body 121 and the first light source mounting plate 122 of the first light source assembly 120. In this case, the opening 124a may correspond to the main opening 124a. For example, each of the plurality of sub openings 124b may be formed between first light sources 123 adjacent to each other.

As the plurality of sub openings 124b are formed, a blocking rate of the light emitted from the second light sources 136 disposed behind the first light source assembly 120 may be effectively reduced, and thus, a sufficient amount of light may be irradiated to the skin to acquire an image having sufficient visibility.

That is, as the plurality of sub openings 124b are formed, the second light sources 136 may be disposed to avoid the provision of the reflected light to the diagnosis area 540, and the blocking of the light emitted from the second light sources 136 by the first light source assembly 120 may be minimized.

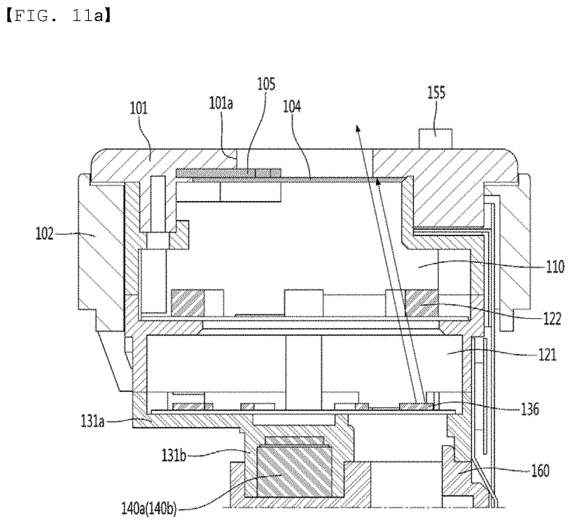

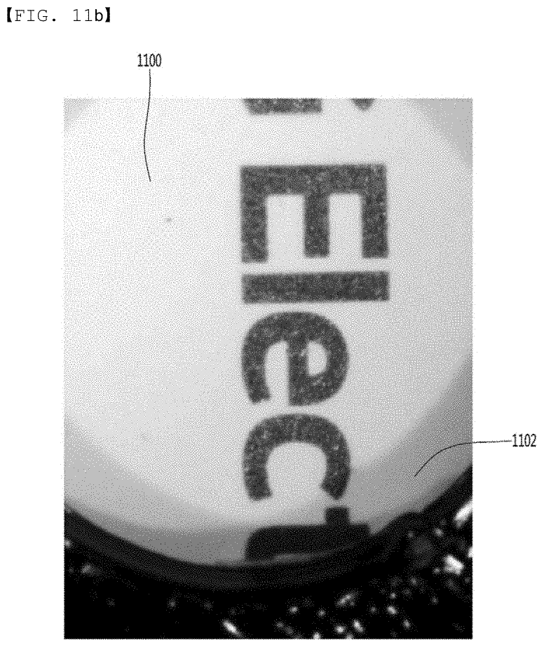

FIG. 10 is a front view illustrating a state in which the front cover and the side cover of the skin diagnosis device are removed according to an embodiment of the present invention.

Referring to FIG. 10, the components in the head 10 may be formed according to the embodiments described above with reference to FIGS. 3 to 9, and thus, when the head 10 is viewed from the front side, at least some of the first light source 123, the second light source 136, the cameras 140a and 140b, and the laser light source 172 may be observed.

That is, light (the ultraviolet light, the white light, the laser light) emitted from each of the first light source 123, the second light source 136, and the laser light source 172 may be irradiated to the skin through the opening 103. In addition, the light reflected from the skin may be incident into the cameras 140a and 140b through the opening 103. Thus, the head 10 having the compact size and provided with the components capable of acquiring the various skin diagnosis information may be provided.

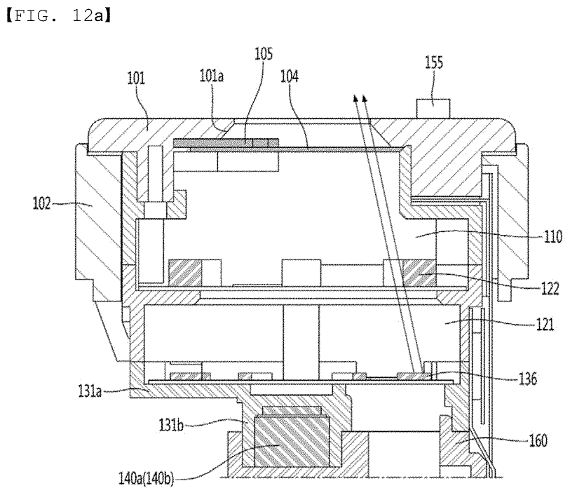

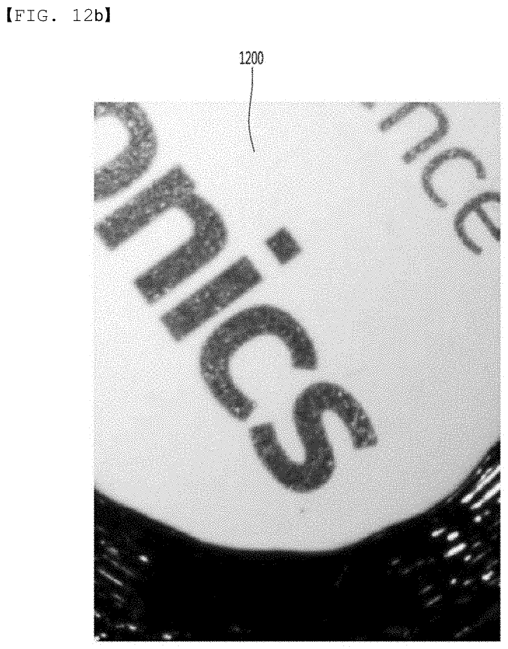

FIG. 11a to 12b are views for explaining a shape of the front cover for preventing the shaded area is provided on the skin diagnosis area of the image acquired by the camera.

Referring to FIGS. 11a and 11b, in order to prevent penetration of external light through a gap between the front cover 101 and the skin or to improve durability of the cover window 104, a radius of the opening 103 may be less than the second distance D2 or the third distance D3 of FIG. 9.

When a boundary surface of the opening 103 is formed to be perpendicular to the skin contact surface of the front cover 101 as illustrated in FIG. 11a, or a radius of the boundary surface is formed to gradually increase toward the skin contact surface, a portion of the light emitted to the area including the edge may be blocked by the rear surface of the front cover 101.

In this case, as illustrated in FIG. 11b, a shaded area 1102 may be generated on a portion including the edge of the opening 103 on the images 1100 acquired by the cameras 140a and 140b.

When the shaded area 1102 is provided in the diagnosis area 540 described above in FIG. 5, the accurate diagnosis information may not be acquired due to a difference in brightness within the diagnosis area 540.

Referring to FIGS. 12a to 12b, a chamfered structure 101b may be formed on an inner circumferential surface (the boundary surface of the opening 103) of the front cover 101 according to an embodiment of the present invention in order to solve the above-described problem. That is, a radius of the inner circumferential surface of the front cover 101 may gradually decrease toward the skin contact surface of the front cover 101.

As the chamfer structure 101b is formed, the blocking of the light emitted to the area including the edge of the opening 103 may be minimized, and the shaded area on the image 1200 acquired by the cameras 140a and 140b may be removed, or the surface area of the shaded area may be minimized.

The shaded area may be removed or minimized to prevent the shaded area from being provided on the diagnosis area 540, thereby acquiring the accurate diagnosis information.

The above-disclosed subject matter is to be considered illustrative, and not restrictive, and the appended claims are intended to cover all such modifications, enhancements, and other embodiments, which fall within the true spirit and scope of the present invention.

Thus, the embodiment of the present invention is to be considered illustrative, and not restrictive, and the technical spirit of the present invention is not limited to the foregoing embodiment.

Therefore, the scope of the present disclosure is defined not by the detailed description of the invention but by the appended claims, and all differences within the scope will be construed as being included in the present invention.

[symbol]

1. A skin diagnosing device comprising:
a main body provided with an input interface for an operation; and
a head connected to one end of the main body, the head including:
a front cover forming a contact surface and including an opening;
a cover window covering the opening of the front cover;
a plurality of ultraviolet light sources disposed behind the front cover and facing the opening of the front cover;
a plurality of visible light sources disposed behind the front cover and facing the opening of the front cover;
a camera disposed behind the plurality of ultraviolet light sources and the plurality of visible light sources and facing the opening of the front cover; and
a laser light source disposed behind the camera and facing the opening of the front cover.

2. The skin diagnosing device according to claim 1, further comprising:
a first inner body in which the plurality of ultraviolet light sources are disposed, the first inner body being coupled to a rear side of the front cover; and
a second inner body in which the plurality of visible light sources are disposed, the second inner body being coupled to a rear side of the first inner body,
wherein the first inner body includes an opening formed in a center of the first inner body, and
each of the plurality of ultraviolet light sources is spaced a first distance from the center of the first inner body.

3. The skin diagnosing device according to claim 2, wherein each of the plurality of visible light sources is spaced a second distance from a center of the second inner body greater than the first distance,
the opening of the first inner body comprises a main opening including the center of the first inner body and a plurality of sub openings formed outside the main opening, and
a radius of the main opening is less than the second distance.

4. The skin diagnosing device according to claim 3, wherein each of the plurality of sub openings is formed between two adjacent ultraviolet light sources among the plurality of ultraviolet light sources.

5. The skin diagnosing device according to claim 3, wherein the second distance is for avoiding light that is emitted from the plurality of visible light sources from being reflected from the cover window to a diagnosis area within an image acquired through the camera.

6. The skin diagnosing device according to claim 2, wherein the second inner body comprises:
a camera opening disposed in a center of the second inner body; and
a laser light source opening disposed between the laser light source and the cover window and disposed between the camera opening and an edge of the second inner body.

7. The skin diagnosing device according to claim 1, further comprising a polarization assembly including:
a first polarizing film disposed in front of a first group of visible light sources among the plurality of visible light sources; and
a second polarizing film disposed in front of a second group of visible light sources among the plurality of visible light sources.

8. The skin diagnosing device according to claim 7, wherein any one of the first polarizing film and the second polarizing film is formed in front one of the camera.

9. The skin diagnosing device according to claim 1, wherein a distance between a central axis of the head in a front and rear direction and the camera is less than a distance between the central axis of the head and the laser light source.

10. The skin diagnosing device according to claim 9, wherein the laser light source is inclined at a predetermined angle with respect to the front and rear direction of the head.

11. The skin diagnosing device according to claim 1, further comprising:
a first inner body in which the plurality of ultraviolet light sources are disposed; and
a second inner body in which the plurality of visible light sources are disposed,
wherein each of the plurality of ultraviolet light sources is spaced a first distance from a center of the first inner body,
each of the plurality of visible light sources is spaced a second distance from a center of the second inner body, and
a radius of an inner circumferential surface of the front cover corresponding to the opening of the front cover is less than each of the first distance and the second distance.

12. The skin diagnosing device according to claim 11, wherein the radius of the inner circumferential surface of the front cover gradually decreases toward the contact surface of the front cover.

13. The skin diagnosing device according to claim 1, wherein the head extends from the one end of the main body at an obtuse angle relative to an extension direction of the main body.

14. The skin diagnosing device according to claim 1, further comprising a gray card disposed between the front cover and the cover window and corresponding to a partial area of the opening of the front cover.

15. The skin diagnosing device according to claim 1, further comprising a moisture measuring sensor disposed inside the head so that a portion of the moisture measuring sensor protrudes to an outside through a sensor hole of the front cover.

16. The skin diagnosing device according to claim 1, wherein the plurality of visible light sources comprise a white light source.

17. A skin diagnosing device comprising:
a head connected to one end of a main body, the head including:
an opening;
a plurality of ultraviolet light sources configured to irradiate ultraviolet light through the opening;
a plurality of visible light sources configured to irradiate visible light through the opening;
a camera configured to receive light reflected from a skin of a user; and
a laser light source configured to irradiate laser light through the opening.

18. The skin diagnosing device according to claim 17, further comprising a moisture measuring sensor protruding through the head and configured to measure a moisture level of the skin of the user upon contact with the skin of the user.

19. The skin diagnosing device according to claim 17, further comprising:
a first inner body in which the plurality of ultraviolet light sources are disposed; and
a second inner body in which the plurality of visible light sources are disposed, the second inner body including:
a laser light source opening corresponding to the laser light source; and
a camera opening spaced from the laser light source opening and corresponding to the camera.

20. The skin diagnosing device according to claim 19, wherein the first inner body is disposed behind the opening,
the second inner body is disposed behind the first inner body, and
the laser light source is disposed behind the second inner body.
